(19) United States
(12) Patent Application Publication
(10) Pub. No.: US 2025/0152284 A1
(43) Pub. Date: May 15, 2025

(54) METHODS, APPARATUS AND SYSTEMS FOR MANIPULATING A MEDICAL DEVICE

(71) Applicants: Canon U.S.A., Inc., Melville, NY (US); CANON KABUSHIKI KAISHA, Ohta-ku, Tokyo (JP)

(72) Inventors: Brian Ninni, Woburn, MA (US); Takahisa Kato, Brookline, MA (US); Fumitaro Masaki, Brookline, MA (US); Charles George Hwang, Wellesley, MA (US); HuaLei Shelley Zhang, Brookline, MA (US); Kiyoshi Takagi, Tokyo (JP)

(21) Appl. No.: 18/835,305

(22) PCT Filed: Feb. 6, 2023
(86) PCT No.: PCT/US23/62067

§ 371 Date: Aug. 1, 2024

Related U.S. Application Data
(60) Provisional application No. 63/307,398, filed on Feb. 7, 2022.

(51) Int. Cl. A61B 34/37; A61B 17/00; A61B 34/00; A61B 34/20; A61B 34/30; A61B 90/00

(52) U.S. Cl. CPC A61B 34/37; A61B 34/71; A61B 90/00; A61B 2017/00314; A61B 2017/00327; A61B 2034/2059; A61B 2034/2061; A61B 2034/301; A61B 2090/064

(57) ABSTRACT

A continuum robot having at least two independently manipulatable bendable section for advancing the robot through a passage, without contacting fragile elements within the passage, wherein the robot incorporates a system, method and apparatus including a ‘relax mode’ capable of relaxing at least one of the bendable sections, allowing the bendable section to conform to the surrounding anatomy.

### CROSS REFERENCE TO RELATED PATENT APPLICATIONS

This application claims priority from U.S. Provisional Patent Application No. 63/307,398, filed on Feb. 7, 2022, in the United States Patent and Trademark Office, the disclosure of which is incorporated by reference herein, in its entirety.

### FIELD OF THE DISCLOSURE

The present disclosure relates generally to medical devices and, more particularly to a continuum robot (also referred to as ‘snake’ or ‘snake system’) applicable to guide interventional tools and instruments, such as endoscopes and other tools, in medical procedures.

### BACKGROUND OF THE DISCLOSURE

A continuum robot or snake includes a plurality of bending sections having a flexible structure, wherein the shape of the continuum robot is controlled by deforming the bending sections. The snake mainly has two advantages over existing robots including rigid links. The first advantage is that the snake can move along a curve in a narrow space or in an environment with scattered objects in which the rigid link robot may get stuck. The second advantage is that it is possible to operate the snake without damaging surrounding fragile elements because the snake has intrinsic flexibility.

In recent years, minimally invasive medical care, with which burden on the patient can be reduced and the quality of life (QOL) after the treatment or inspection can be improved, has been attracting attention. A surgery or inspection using an endoscope is a typical example of minimally invasive medical care. For example, a laparoscopic surgery is advantageous over a conventional abdominal surgery in that it can be performed with a smaller surgical wound, which results in a shorter stay in the hospital and less damage to the appearance.

Endoscopes used for the minimally invasive medical care are roughly divided into rigid endoscopes and soft endoscopes. With a rigid endoscope, although clear images can be obtained, the direction in which an observation target can be observed is limited. In addition, when the rigid endoscope is inserted into a curved organ, such as the esophagus, large intestine, or urethra, an insertion portion of the rigid endoscope presses the organ and causes pain for the patient. In contrast, a soft endoscope includes an insertion portion formed of a bendable member, so that a large area can be observed in detail by adjusting the bending angle of the distal end of the endoscope. In addition, by bending the insertion portion along an insertion path, burden on the patient can be reduced. When the number of bendable portions is increased, the endoscope can be inserted to a deep area of the body without causing the endoscope to come into contact with tissue even when the insertion path has a complex curved shape.

Accordingly, soft endoscopes having a plurality of bendable portions have been widely researched and developed.

However, while navigating through the airway, the endoscopes pose does not necessarily match the shape of the passageway. This mismatch can cause the endoscopes to experience friction against the airway wall. This friction can cause the endoscopes to twist or prolapse, making navigation more difficult or impossible. In addition, this can impart a lot of force into the anatomy, and potentially cause trauma to the subject. This phenomena is not unique to multi-section robotic endoscopes, but it could be a more frequent occurrence, since it can take more complex shapes.

Various related art disclosures in the field include: US 2015/0142013 which discloses releasing tension from the continuum robot pull wires with a button/command for the continuum robot shape to conform to the anatomy. However, there are issues with disclosed method, including: 1) When insertion motion is combined with this releasing, the continuum robot shape would deviate from the anatomy shape more than desired; and 2) When the actuator pushes the wires along with pulling, it is difficult to release both compression and tension forces on the wires only by releasing tension on the wires.

As such, the subject innovation introduces a solution to this quandary.

### SUMMARY

Thus, to address such exemplary needs in the industry, the presently disclosed apparatus teaches a robotic apparatus comprising: a continuum robot including a plurality of bending sections including a distal bending section and a proximal bending section wherein each of the bending sections are bent by at least one wire, as well as an actuator for moving the at least one wire, and a controller for controlling the actuator based on instructions from an end user, wherein the actuator includes at least one motor for moving the at least one wire, and at least one sensor in communication with the wire for detect a force on the at least one wire, and wherein the controller further comprises a relax mode for reducing tensile forces in the at least one wire and relaxing at least one of the bendable sections by using force feedback control of the at least one motor based on information from the at least one sensor.

In other embodiment, the relax mode further comprises reducing compression forces in the at least one wire and relaxing at least one of the bendable sections. Furthermore, another embodiment includes reducing both compression and tensile forces is independent of actuator mechanical structure and accomplished by using the force feedback control to release forces on the at least one wire.

In other embodiments, the relax mode is enacted based on a force measurement data on the at least one wire. Additionally, relax mode may be passively enacted when the at least one sensor reaches a threshold force measurement. Alternatively, relax mode can be activated or deactived by an end user. This may be accomplished when the end user engages a button to activate and/or deactive relax mode.

In another embodiment, the end user may engages a button to activate relax mode, and after a predetermined time interval, relax mode is automatically deactivate.

In yet additional embodiments, the controller does not reflect the end user's bending command for the bendable sections in relax mode.

In other exemplary embodiments, the apparatus further comprises an insertion stage to move the continuum robot in a longitudinal direction with respect the continuum robot, wherein movement of the insertion stage is controlled by the controller by instructions from the end user. It is further contemplated that relax mode is enacted only when the insertion stage is static.

In another embodiment, relax mode may be enacted for inserting and/or removing the plurality of bending sections.

The subject innovation, further teaches a continuum robot control system comprising: a continuum robot including a plurality of bending sections including a distal bending section and a proximal bending section wherein each of the bending sections are bent by at least one wire; an actuator for moving the at least one wire; and a controller for controlling the actuator based on instructions from an end user, wherein the actuator includes at least one motor for moving the at least one wire, and at least one sensor in communication with the wire for detect a force on the at least one wire, and wherein the controller further comprises a relax mode for reducing tensile forces in the at least one wire and relaxing at least one of the bendable sections by using force feedback control of the at least one motor based on information from the at least one sensor.

These and other objects, features, and advantages of the present disclosure will become apparent upon reading the following detailed description of exemplary embodiments of the present disclosure, when taken in conjunction with the appended drawings, and provided paragraphs.

### BRIEF DESCRIPTION OF THE DRAWINGS

Further objects, features and advantages of the present innovation will become apparent from the following detailed description when taken in conjunction with the accompanying figures showing illustrative embodiments of the present innovation.

FIG. 1 is a block diagram of an exemplary bendable medical device incorporating various ancillary components, according to one or more embodiment of the subject apparatus, method or system.

FIG. 2 illustrates a kinematic model of the subject continuum robot, according to one or more embodiment of the subject apparatus, method or system.

FIG. 3 provides a detailed illustration of the subject continuum robot, according to one or more embodiment of the subject apparatus, method or system.

FIG. 4a and FIG. 4b are images of a target area in the lungs, in connection with a pathway of the subject continuum robot, according to one or more embodiment of the subject apparatus, method or system.

Throughout the Figures, the same reference numerals and characters, unless otherwise stated, are used to denote like features, elements, components or portions of the illustrated embodiments. In addition, reference numeral(s) including by the designation “′” (e.g. 12′ or 24′) signify secondary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

### DETAILED DESCRIPTION OF THE DISCLOSURE

In the subject disclosure, Applicant will first detail the mechanism of a continuum robot, followed by the continuum robot ‘relax’ mode, and functionality methods, as well as the systems and procedures associated with the continuum robot and said ‘relax’ mode.

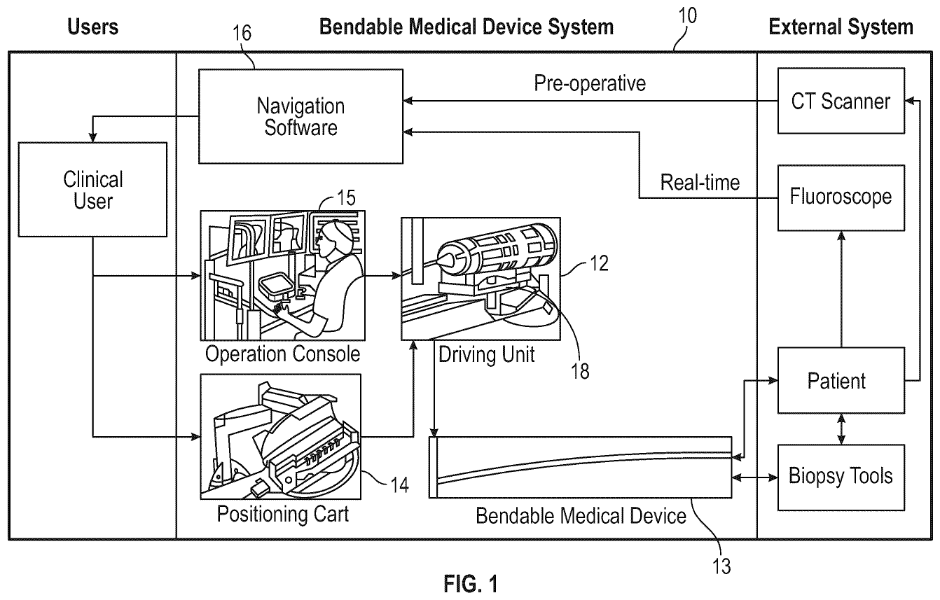

FIG. 1 is a system block diagram of an exemplary bendable medical device system 10 incorporating various ancillary components intended to amass a complete medical system. The bendable medical device system 10 comprises an actuator or driving unit 12 (also referred to herein as a ‘driver’) for driving the wires, and having a base stage 18, a bendable medical device 13, a positioning cart 14, an operation console 15 (also referred to herein as ‘controller 15’), having push-button, thumbstick, and/or joystick operational console 15, and navigation software 16. The exemplary bendable medical device system 10 is capable of interacting with external system component and clinical users to facilitate use in a patient.

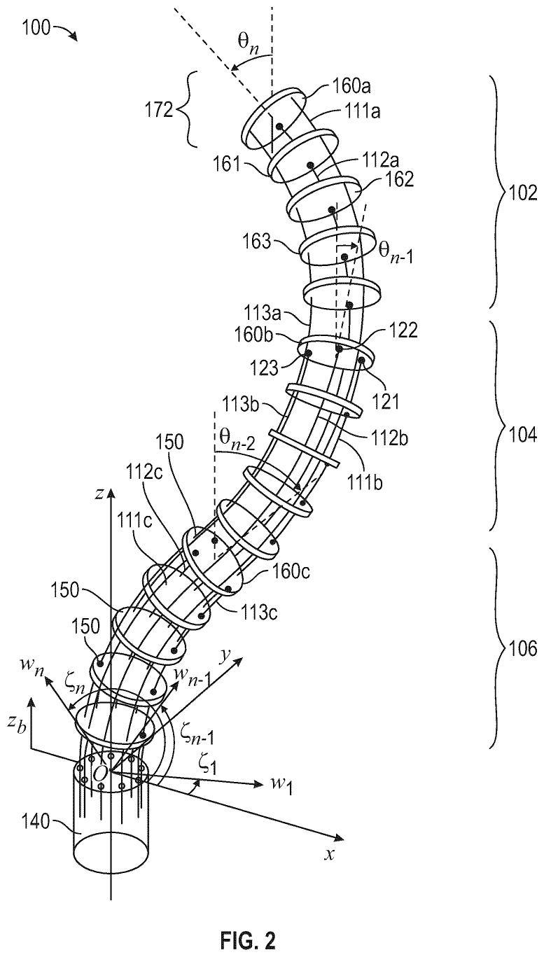

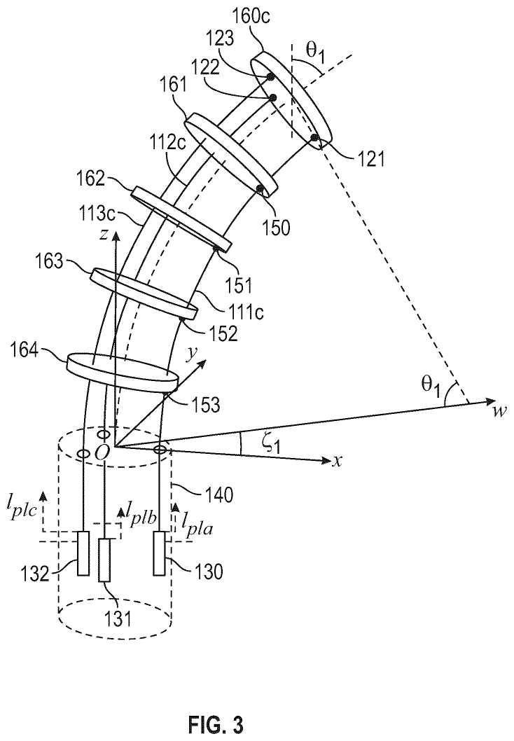

FIG. 2 illustrates a continuum robot 100 that is capable of a plurality of bends, with FIG. 3 providing an enlarged view of the proximal end of the robot 100.

As shown in FIG. 2, the continuum robot 100, comprises wires 111b, 112b and 113b, which are connected to connection portions 121, 122 and 123, respectively, found on an end disc 160b, for controlling the middle bending section104. Additional wires (3 for each of the other bendable sections 102 and 106) 111a, 1110, 112a, 112c, 113a, 113c, are attached at the distal ends of each bendable section 102 and 106, to the respective end disc 160a and 160c.

As each bending section is operated similarly, we will focus on one bending section, here the middle bending section 104, to explain the mechanism. The posture of the bending section 104 is controlled by pushing and pulling the wires 111b to 113b by using actuators 130 to 132 disposed in a robot base 140. (Note—In the interest of clarity, only actuators for the three wires 1110, 112c, 113c have been show in FIG. 3, additional actuators for the remaining 6 wires are contemplated in this innovation.)

Moreover, the robot base 140 of the continuum robot 100 is disposed on a base stage 18 (See FIG. 1) and can be moved by the base stage 18 in the longitudinal direction. Thus, it is possible to advance and retard the robot 100 into a target structure by advancing and retarding the base stage 18.

An operational console 15 (see FIG. 1) indicates a driving amount to the base stage 18 and, independently, to the actuators 130 to 132. Throughout this disclosure, the operational console 15 may also be described or eluded to as a control system or controller. The operational console 15 may include dedicated hardware including a field-programmable gate array (“FPGA”) and the like; or may be a computer including a storage unit, a work memory, and a central processing unit (“CPU”). In the case where the operational console 15 is a computer, the storage unit may store a software program corresponding to an algorithm of the control system (described below) and the central processing unit expands the program in the work memory, executes the program line by line, and thereby the computer functions as the operational console 15. In either case, the operational console 15 is communicably connected to the base stage 18 and the actuators 130 to 132, and the operational console 15 send signals representing the driving amount and configuration to these control targets, which are imputed by an end user through push buttons, joystick or the like.

The continuum robot 100 includes multiple wire guides 161 to 164 situated throughout each bending section, and moreover detailed in FIG. 3 for proximal bending section 106. The wire guides 161 to 164 are shown here guiding the wires 1110, 112c and 113c, and for providing structural integrity to the bending section 106. As before and for the sake of redundancy, we have elected to detail the components of the proximal bending section 106 in FIG. 3, with the understanding that the remaining bending sections 102 and 104, function in a similar fashion with similar elements. The wire guides 161 to 164 each contain a wire through 150-153 for each wire 1110-113c. For ease of illustration, FIG. 3 only depicts the wire through 150-153 for a single wire 111c. Alternatively, a method of discretely arranging the plurality of wire guides, a continuum robot 100 having a bellows-like shape or a mesh-like shape may be utilized, wherein the wire guides 161-164 are fixed to their respective wires 111a-113a.

With respect to FIGS. 2 and 3, the definitions of symbols are as follows: l_{d}=the length of the central axis a bending section; θₙ=the bending angle of the distal end; ζₙ=the rotational angle of the distal end; ρₙ=the radius of curvature of a bending section.

At detailed above, the continuum robot in this embodiment includes at least one distal bending section with robotic insertion and removal of the continuum robot from the target.

At its most basic level, the subject innovation incorporates a ‘relax’ mode, which allows the shape of the continuum robot to conform to the airway or other anatomy surrounding the robot. This will reduce the interaction between the continuum robot of the wall of the anatomy, and therefore reduce the amount of times and severity of the twisting and prolapsing of the robot. It will also reduce the forces being applied against the anatomy. The advantage of relaxing specific sections of the robot is that it can better align with the surrounding structure and reduce the interaction with the anatomy along each section.

As stated above, the continuum robot system 10 in this disclosure comprises a continuum robot 100, an actuator or driving unit 12, an insertion stage 18 and a controller 15. The continuum robot 100 includes distal and proximal ends with a tubular body, and at least one bending section 102. The bending section(s) 102, 104 and 106, include driving wires 111-113 terminating at the distal end of each bending section, typically at an end disc 160. A proximal end of the driving wires 111-113 are connected to the actuators 130-132. The actuators include motors and a linear motion mechanism. The linear motion mechanism is connected with the motors on one end and the proximal end of the driving wires on the other end. Via the linear motion mechanism, the actuator can push and pull the driving wires to bend the continuum robot with the motors. The motors are controlled by the controller 15 (also referred to herein as ‘operation console’). An assembly of the continuum robot 100 and the actuator 12 is mounted on the insertion stage 18. The insertion stage 18 includes a linear motor to move the assembly of the continuum robot 100 and the actuator 12 in the direction of the insertion and removal for the clinical workflow. The insertion stage 18 is also controlled by the controller 15.

The current implementation measures forces on the driving wires 111-113 with force sensors 170 at the junction between the robot driving wires 111-113 and the actuators 130-132. Each drive wire 111-113 has its own sensor 170. To reduce compression and tension forces on the wires 111-113 at the same time, the controller 15 uses a force feedback control, which measures the forces on the wires 111-113 with these sensors 170, and generates the motor commands with a robot control response properties to reduce the forces on the wires 111-113. Specifically, in the current specific implementation, the controller 15 will move each wire independently in the relax mode without considering catheter's kinematics to create each pose.

### Embodiment 1: Relax Mode in Normal Navigation

In the normal navigation through the anatomy, the operator commands to bend the continuum robot and to insert or remove the continuum robot with a controller/joystick located on the controller. The controller accepts these commands and actuates the actuator and the insertion unit to execute the intended motion.

However, in some situation, the operator might insert the robot with the shape that does not conform to the anatomy. When the operator continues to insert the robot in this situation, the robot may collide with the anatomy. While doing so, it starts deforming the anatomy, while also itself being deformed by the anatomy. This mutual deformation would make the robot navigation difficult, resulting in reduced bending motion and prolapsing of the proximal part of the robot.

Wire force reduction with force feedback control, i.e., the relax mode, would mitigate the shape mismatch between the robot section and anatomy, and avoid the deformation of both the robot and anatomy.

This relax mode with the force feedback control has benefit to maintain all control status over conventional mechanical disengagement of wires to release the forces. During the relax mode in this innovation, the controller keeps all motor current position values with the initial physical zero position of the wires and motors. Therefore, the system can switch the operation mode accurately and easily between the relax mode and the normal bending mode.

Also, with this method, we can achieve the relax mode for both compression and tensile forces on wires even when the actuator include linear motion mechanism to convert rotational motion of the motors to push-pull motion to move the wires. This motion mechanism is not usually backdrivable due to its internal friction and gear ratio of motion conversion even when the controller can reduce holding torque on the motors.

This relax mode can be initiated/exited at the users request, with the push of a button. When the system initiates the relax mode, the controller first suspends the insertion stage motion and suspends acceptance of bending commands for the robot. Then, the controller executes the wire force reduction with force feedback control. Relax mode will attempt to reduce the forces in all driving wires by moving the actuator motor that the drive wire is attached to. Since each section connects three driving wires at the distal end, there is an optimal position for each wire since the motion of one wire will affect the forces of the other wires in that section. Also, since all sections are coupled by the inner and outer walls, this interaction also extends to the other 6 wires as well. Each section of the robot undergoes an “auto-tune” procedure (once, after manufacturing) where the interaction between each of the 9 driving wires is measured to create a calibration file with parameters that improve the efficiency of the relaxation algorithm.

In another design, the controller can indicate to the user to initiate the relax mode when the system detects this type of deformation by monitoring variation of the forces on the wires with the force sensors. This can be done with a user-defined force threshold or pre-determined metrics computed from the force measurement data with the force sensor.

In another design, the user push the button to activate the relax mode. Then the controller stays the relax mode for the predetermined time period, which is defined either as a default value or by the user. After the predetermined time period, the controller disable the relax mode automatically and return to the normal operation mode.

In another design, the controller does not accept the user command to activate the relax mode during the insertion stage is moving.

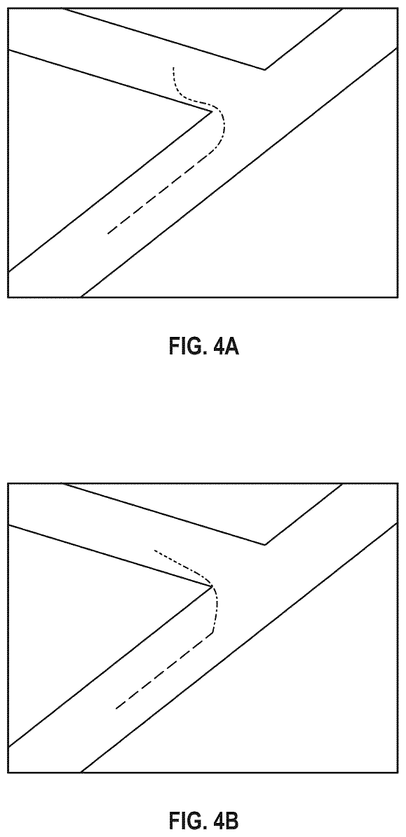

By way of examples, an exemplary navigation workflow is partially depicted in FIGS. 4a and 4b, and could include: 1—Navigating through the airway using FTL; 2—Monitoring force readouts to indicate high forces in certain wires; 3—Cease controlling/manipulating the stage/catheter (see FIG. 4a); 4—Press button to enter relax mode; 5—Wait for the forces to reduce and stabilize (see FIG. 4b); 6—Press button to exit relax mode; and 7—Continue navigating using FTL.

It is further contemplated that the each section of the robot can be relaxed independently from each other, both manually and automatically. For example, the middle and proximal section might be in relax mode while the tip is still controllable. In this way, interaction with the anatomy from FTL will not exist, and these section might get “pulled” in the tip direction due to the cross talk. Additionally, the user can define varying degrees of conformity, and it can be unique to each section of the robot.

### Embodiment 2: Auto Relax Function

During the navigation in embodiment 1, the system in this embodiment can initiate the relax mode automatically without the operator's engaging the button for the relax mode. Here the sensors can monitor input and when a pre-determined threshold/force condition is reached, the system automatically initiates relax mode. The pre-determined threshold/force condition would be a higher threshold force value than the threshold force value to recommend user to use the relax mode. Also, the pre-determined threshold/force condition can be based on force variation speed or the force difference between the measured forces and the force calculated necessary to pull/push amount with ideal kinematic of the catheter. When the system automatically initiates relax mode with the pre-determined threshold/force condition, the system also informs the operator that it entered the relax mode automatically, and may limit manipulation of the robot.

### Embodiment 3: Manual Insertion

The insertion stage in this third embodiment includes a motorized slider and a manual slider. The actuator is attached on the manual slider while the manual slider is mounted on the motorized slider. At the beginning of the procedure, the operator may set the manual slider position to the initial (zero) position manually. Then, the operator sets the position of the motorized slider to the initial (zero) position by instructing the controller. After the initialization of both manual and insertion sliders, the operator attaches the catheter to the actuator on the manual slider. Also, the operator sets the endoscope camera in the tool channel of the robot. The controller asks the operator whether the controller can start the manual insertion mode.

With the operator's instruction, the controller starts manual insertion mode, wherein the controller activates the relax mode, which suspends the motorized slider motion and acceptance of bending commands, then executes the wire force reduction with force feedback control. At the same time, the controller display the instruction to the operator for manual insertion with the manual slider. The operator manually inserts the robot with the manual slider. During this manual insertion, the operator checks the endoscope image with the endoscope camera. In addition, the controller monitors the manual slider position with an encoder in the manual slider. Once the operator inserts the robot to the desired degree (i.e., the first bifurcation of the lung) by checking the endoscope image, the operator instruct the controller to finish the manual insertion mode. Finally, the controller moves the current state to the navigation mode.

A typical exemplary workflow example may include: Operator instructs to enter manual insertion mode, followed by controller activates manual insertion mode, wherein in manual insertion mode, the controller activates relax mode and continues to read the manual slider position with the encoder, and finally the operator inserts the robot with the manual slider.

### Embodiment 4: FTL Smoothing

Due to the robot having multiple bending sections, a unique control mode called Follow-the-Leader (FTL) is employed. In this control mode, the user only commands bending motion of the most distal bending section, while all preceding sections are automatically controlled by the controller and follow the leaders (most distal bending section) path. During insertion, the controller guides the preceding section's pose to match the pose of it's subsequent section at that point along the insertion path. When relax mode is used, the controller updates the FTL algorithm to guide the sections towards the newly relaxed pose, and smooths the motion transition during insertion.

Another control mode, reverse FTL (rFTL) is used while retracting the robot. This mode automatically controls all sections to retrace the pose from the same position during insertion. Again, when relax mode is used, this rFTL algorithm will update the algorithm to refer to the relaxed poses at certain positions, and smooth the transition to that pose.

In addition, there are additional conditions which will benefit from relax mode, including:

Robot Twisting—As the robot rubs along the wall, it might roll/twist about its longitudinal axis due to friction. When this occurs, the controllability of the robot will be compromised because it will not long bend in the direction that the system is commanding/directing it to. Relaxing here will reduce the interaction with the anatomy, and the robot can naturally return to it's initial orientation. To achieve this, the system needs to be aware that the robot is twisting. One way of doing this is with the sensors at the tips of the robot (6 degrees of freedom sensors). Alternatively, with a 5 degrees of freedom sensor the system can detect if the robot is not bending in the expected direction.

Robot Prolapsing-Depending on the scenario, collision with the anatomy might not appear as high forces along the driving wires if the interaction is “helping” the robot bend in the intended direction. However, this often occurs when the desired bending angle is very large. It is possible that situation might occur in conjunction with prolapsing, where a portion of the robot collapses. When this happens, continuing to insert the robot will increase the severity of the prolapse, rather than propel the tip forward. This scenario can be identified by the user, since the camera in the robot will not show any motion. It can also be detected by the system with a position sensor at the tip. When this is detected, it can indicate to the user that it is in a prolapse state and relaxing can help release it by pulling the robot away from the wall, and potentially helping pull the prolapsed portion in the desired direction.

Examples of the workflow covering the prior two scenarios may include: Navigating through the airway using FTL; Software detects tip position does not change in the way the controller expects; Software indicates to user that they should relax; User agrees; Controller relaxes robot; User exits relax mode; and Continue navigating using FTL.

Robot Retraction-Currently, our system uses the rFTL algorithm to retract the path that the robot tool during insertion. However, due to changes in anatomy shape due to breathing motion, or simply due to a less than ideal path taken during insertion, this might still cause collision with the anatomy. If relax mode were to be using while retracting, any collision with the anatomy will be detected and the robot pose can immediately adjust to minimize future impacts.

Phase in breathing cycle—At the extremes of the breathing cycle, the shape of the airway might looks very different. In these scenarios, the robot might exert a large force upon the anatomy if the shape no longer matches the shape it was initially. If the breathing cycle can be detected, the system can enter relax mode during portions where large interaction is expected (or previously detected) and exit relax mode after exiting that phase. One way the robot might be able to detect the breathing cycle is using a position sensor in the tip, and tracking its displacement.

Tool Exchange—It might be difficult to remove/insert a tool through the tool channel if the robot has a large curvature. In this scenario, relaxation can detect the force imparted by the tool and relax its pose to accommodate the passing. The system can store the pre-relax pose, and return to it after tool exchange is complete.

Longitudinal forces on the Tip (Head on collision with anatomy)/Compression—The robot might also collide with the anatomy head-on at the tip. One scenario where this might occur is during stage insertion. This can be detected with contact or force sensors on the tip. Since this will put the robot into compression, sensors at other locations might be able to identify this collision. For example, without our current implementation, compressive forces will cause a delta force in the same direction for all sensors in that section, which can be detected.

In some cases, the change in pose of the robot might not release it from this collision if the compression force is too large. In this case, another way of reducing the interaction with the anatomy would be from retracting the stage (or inserting, in some situations).

### Alternative Mode Initiations:

A separate “emergency button” can trigger relax mode, in addition to a button on the controller or user interface.

Instead of toggling relax mode on/off, the user can hold the button and relax mode will only be active while that button is pressed.

The force thresholds for indication or auto-relax can vary based on the state of robot and scenario. For example, a large force is expected if the robot is at a large output angle. In another scenario, the robot might need to intentionally press against the airway to aim in a certain direction, for example to aim at the lesion in targeting mode.

Similar to the above, the force thresholds might vary for each section of the robot. In one scenario, the user might want a higher threshold for the tip section since they can control it.

Continuing that train of thought, each drive wire can have its own threshold based on the state. For example, a wire is more likely to fail in compression than tension, so the force threshold might be lower when a wire is being pushed. Additionally, each wire can enter relax mode individually.

Since the force sensors are at the proximal portion of the driving wires, a force applied to any location along the length of the robot can be detected, but the location can't be known. Using other sensors (like shape, haptic, or position) along the length of the body can help identify more accurately where the force is occurring.

The system can also exit relax mode automatically, based on certain criteria. For example, when the force falls below a threshold, the robot reaches a desired pose, or after a certain period of time.

The system can analyze a medical image (for example, the pre-OP planning CT) or the endoscope view, and pre-emptively identify areas that might be difficult and could benefit from relaxation. Then, it can inform the user as they approach/enter this zone that they should relax. Its position relative to the difficult portion of the airway can be determined with a position sensor or by measuring the insertion distance so far (via the insertion stage). One way the software might be able to rate an airway is with a ‘difficult score’ (see PCT/US21/44787). Similarly, by comparing the shape of the robot (for example, with shape sensors or forward kinematics) to the segmented airway, the system can actively prepare the pose of the robot to match the airway.

### Alternative Method of Conforming to Airway:

Release the driving wires and let the catheter naturally relax—For navigation continuity, the controller needs to know the pose of the robot at each step of the advancement. This is necessary for the FTL function (since each sections follow the pose of the subsequent distal sections) and for rFTL (since the pose of the catheter pose gets retracted while retracting). In addition, this algorithm include a “smoothing” function to reduce large motion while they are active.

Since this method is performed passively, the system needs to determine the pose after relaxation. One method if by having shape or position sensors in each section, and the pose can be measured from these means. Another method is by having relative or absolute encoders on the drive wires, and measuring the position of the drive wires.

### Complementary Embodiments

Continue to bend in the direction it moves during relaxation to pull away from the wall, rather than simply rest against it.

While the present disclosure has been described with reference to exemplary embodiments, it is to be understood that the present disclosure is not limited to the exemplary embodiments described.

1. A robotic apparatus comprising:
a continuum robot including a plurality of bending sections including a distal bending section and a proximal bending section wherein each of the bending sections are bent by at least one wire;
a driver that drives the wire; and
an operational console that controls a movement of the driver, based on an input,
wherein the operational console includes at least one motor for moving the at least one wire, and at least one sensor in communication with the wire for detecting a force on the at least one wire, and
wherein the operational console further comprises a relax mode for reducing tensile or compression forces in the at least one wire for relaxing at least one of the bendable sections by using force feedback control of the at least one motor based on information from the at least one sensor.

2. The apparatus of claim 1, wherein relax mode further comprises reducing tensile or compression forces in the at least one wire for relaxing at least one of the bendable sections.

3. The apparatus of claim 2, wherein reducing both compression and tensile forces is independent of actuator mechanical structure and accomplished by using the force feedback control to release forces on the at least one wire.

4. The apparatus of claim 1, wherein the relax mode is enacted based on a force measurement data on the at least one wire.

5. The apparatus of claim 4, wherein the relax mode may be passively enacted when the at least one sensor reaches a threshold force measurement.

6. The apparatus of claim 1, wherein the relax mode can be activated or deactivated by an end user.

7. The apparatus of claim 6, wherein the end user engages a button to activate and/or deactivate relax mode.

8. The apparatus of claim 6, wherein the end user engages a button to activate relax mode, and after a predetermined time interval, relax mode is automatically deactivate.

9. The apparatus of claim 1, wherein the operational console does not reflect the end user's bending command for the bendable sections in relax mode.

10. The apparatus of claim 1, further comprising a base stage to move the continuum robot and driving unit in a longitudinal direction, wherein movement of the base stage is controlled by the operational console by instructions from the end user.

11. The apparatus of claim 10, wherein relax mode is enacted only when the base stage is static.

12. The apparatus of claim 1, wherein relax mode may be enacted for inserting and/or removing the plurality of bending sections.

13. A method for controlling a robotic apparatus:
the robotic apparatus comprising:
a continuum robot including a plurality of bending sections including a distal bending section and a proximal bending section wherein each of the bending sections are bent by at least one wire;
a driver that drives the at least one wire;
an operational console that controls a movement of the drive, based on an input; and
a sensor in communication with the at least one wire for detecting a force on the at least one wire,
wherein the method comprises:
receiving an input from a user to the operational console;
sending a signal from the operational console to the driver to drive the at least one wire to manipulate the continuum robot;
monitoring the sensor to detect a force on the sensor;
engaging a relax mode for reducing tensile or compression forces in the at least one wire for relaxing at least one of the bendable sections by using force feedback control of the at least one motor based on information from the sensor.

14. The control system of claim 13, wherein relax mode further comprises reducing tensile and compression forces in the at least one wire for relaxing at least one of the bendable sections.

15. The control system of claim 14, wherein reducing both compression and tensile forces is independent of actuator mechanical structure and accomplished by using the force feedback control to release forces on the at least one wire.

16. The control system of claim 13, wherein the relax mode is enacted based on a force measurement data on the at least one wire.

17. The control system of claim 13, wherein the relax mode can be activated or deactived by an end user.

18. The control system of claim 17, wherein the end user engages a button to activate relax mode, and after a predetermined time interval, relax mode is automatically deactivate.

19. The control system of claim 13, wherein the controller does not reflect the end user's bending command for the bendable sections in relax mode.

20. The control system of claim 13, further comprising an insertion stage to move the continuum robot in a longitudinal direction with respect the continuum robot, wherein movement of the insertion stage is controlled by the controller by instructions from the end user.

21. The control system of claim 13, wherein relax mode may be enacted for inserting and/or removing the plurality of bending sections.
